(19) United States
(12) Patent Application Publication
(10) Pub. No.: US 2024/0238219 A1
(43) Pub. Date: Jul. 18, 2024

(54) CURCUMIN AND TURMERONE COMPOSITIONS & METHODS OF USE THEREOF

(71) Applicant: Franco Cavaleri, Surrey (CA)

(72) Inventor: Franco Cavaleri, Surrey (CA)

(21) Appl. No.: 18/619,314

(22) Filed: Mar. 28, 2024

Related U.S. Application Data
(63) Continuation-in-part of application No. 17/170,956, filed on Feb. 9, 2021.
Application No. 17/170,956 is a continuation of application No. 16/111,502, filed on Aug. 24, 2018, now Pat. No. 10,945,970.
Application No. 16/111,502 is a continuation-in-part of application No. PCT/CA2018/050275, filed on Mar. 8, 2018.
(60) Provisional application No. 63/458,336, filed on Apr. 10, 2023.
(60) Provisional application No. 62/469,554, filed on Mar. 10, 2017.

(51) Int. Cl. A61K 31/12; A61K 9/48; A61P 3/06; A61K 36/9066

(52) U.S. Cl. CPC A61K 31/12; A61K 9/485; A61K 9/4858; A61K 9/4866; A61P 3/06; A61K 36/9066; A61K 2300/00

(57) ABSTRACT

Compositions that include concentrated amounts of curcumin II and/or curcumin III, along with concentrated amounts of turmerones, are disclosed. The compositions may be useful for increasing LDL receptor levels in a plurality of cells. In addition, the compositions may be useful for decreasing MSK1 levels in a plurality of cells. The compositions may also be used to increase expression of brain derived neurotrophic factor (BDNF) in a plurality of cells.

### CROSS-REFERENCE TO RELATED APPLICATIONS

This application claims priority to U.S. provisional patent application Ser. No. 63/458,336, filed on Apr. 10, 2023. This application is also a continuation-in-part of U.S. patent application Ser. No. 17/170,956, filed on Feb. 9, 2021, which is a continuation application of U.S. patent application Ser. No. 16/111,502 (now U.S. Pat. No. 10,945,970), filed on Aug. 24, 2018, which is a continuation-in-part application of PCT application serial number PCT/CA2018/050275, filed on Mar. 8, 2018, which claims priority to, and incorporates by reference, U.S. provisional patent application 62/469,554, filed on Mar. 10, 2017.

### FIELD OF THE INVENTION

The field of the present invention relates to certain curcumin- and turmerone-containing compositions and methods of use thereof. Such compositions can be used to increase low-density lipoprotein cholesterol (LDL) receptor expression levels and thereby lower LDL levels in a plurality of cells or subject. In addition, the field of the present invention relates to certain curcumin- and turmerone-containing compositions, which can be used to modulate MSK1 production and thereby ameliorate a variety of health conditions. Still further, the field of the present invention relates to certain curcumin- and turmerone-containing compositions, which can be used to modulate BDNF (brain derived neurotropic factor) expression levels.

### BACKGROUND OF THE INVENTION

The health benefits of curcumin and turmerones, particularly from whole turmeric extract, are known and have been demonstrated by researchers in recent years. However, several challenges continue to exist, with respect to the formulation of curcumin- and turmerone-based pharmaceuticals and dietary supplements. More specifically, the most common source of curcumin, the Indian spice turmeric (a member of Zingiberaceae), does not contain a sufficient amount of curcumin (or turmerones) to provide an efficacious dose to a subject. In fact, the therapeutic benefits provided by natural curcumin extracts have been relatively modest, very inconsistent, and not well understood. Accordingly, there is a continuing need for improved curcumin- and turmerone-based formulations, which address these current challenges.

The present invention, as described further below, addresses many of the foregoing challenges.

### SUMMARY OF THE INVENTION

According to certain aspects of the present invention, compositions that include a combination of curcumin extract and turmerones are provided. In certain embodiments, the compositions include a curcumin extract component that comprises concentrated and elevated amounts of curcumin II and/or curcumin III, along with concentrated and elevated amounts of turmerones. For example, in certain embodiments, the composition will comprise at least 30% by weight curcumin II; 30% by weight curcumin III; or 30% by weight curcumin II and curcumin III. In other embodiments, the composition will comprise at least 50% by weight curcumin II; 50% by weight curcumin III; or 50% by weight curcumin II and curcumin III. In addition, such compositions include at least 5% by weight of turmerones, such as at least 8%, 10%, 15%, 20%, 30%, or 40% of turmerones. The invention provides that such turmerones may include one or more of alpha-turmerone, beta-turmerone, and/or ar-turmerone (aromatic turmerone). The invention further provides that such curcumin- and turmerone-enriched compositions may be formulated as a capsule, pill, tablet, granule, solution or a suspension in an aqueous or non-aqueous liquid, oil-in-water or water-in-oil emulsion, elixir, syrup, paste, or dry powder.

According to additional aspects of the present invention, methods for increasing low-density lipoprotein cholesterol (LDL) receptor expression levels in a plurality of cells are disclosed (and, by extension, methods of reducing LDL levels in the cells). In certain embodiments, the methods comprise providing to the cells a curcumin- and turmerone-enriched composition described herein.

According to still further aspects of the present invention, methods for modulating mitogen- and stress-activated protein kinase 1 (MSK1) in a plurality of cells are disclosed, which comprise providing the cells with a curcumin- and turmerone-enriched composition described herein.

According to yet further aspects of the present invention, methods for modulating brain derived neurotrophic factor (BDNF) in a plurality of cells are disclosed, which comprise providing the cells with a curcumin- and turmerone-enriched composition described herein. BDNF plays an important role in neuronal survival and growth (and brain plasticity), which are critical for memory and learning activities. In addition, BDNF is believed to play an important role in certain neurodegenerative diseases.

The above-mentioned and additional features of the present invention are further illustrated in the Detailed Description contained herein.

### BRIEF DESCRIPTION OF THE FIGURES

FIG. 1 is a bar graph summarizing the results of the Example described below, showing the ability of certain curcumin- and turmerone-containing compositions to modulate BDNF (brain derived neurotrophic factor) expression levels.

### DETAILED DESCRIPTION OF THE INVENTION

The following will describe, in detail, several preferred embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

According to certain preferred embodiments, the present invention includes certain curcumin- and turmerone-enriched compositions (and methods of using such compositions). In certain embodiments, the compositions of the present invention include a curcumin extract and turmerones. In certain preferred embodiments, the present invention includes certain compositions that comprise a curcumin extract component which comprises elevated and concentrated levels of (1) curcumin II (relative to the amount of curcumin II found in natural curcumin extract); (2) curcumin III (relative to the amount of curcumin III found in natural curcumin extract); or (3) a combination of curcumin II and curcumin III (relative to the amounts of curcumin II and curcumin III found in natural curcumin extract). Notably, the LDL receptor-modulating compositions described herein will preferably exclude curcumin I. The invention provides that such compositions can be used to increase low-density lipoprotein cholesterol (LDL) receptor expression levels in a plurality of cells (which, in turn, results in lower LDL levels in a subject and ameliorates a variety of associated health conditions and/or impart one or more associated health benefits).

In addition, the invention provides that certain curcumin III enriched compositions described herein may be used to modulate mitogen- and stress-activated protein kinase 1 (MSK1), which is a nuclear kinase that plays a significant role in transcription regulation. The invention provides that curcumin III (and not curcuminoids I and II) can be used to selectively and efficaciously inhibit cytoplasmic and nuclear MSK1 production, the inhibition of MSK1 serine³⁷⁶ phosphorylation, and inhibition of the recruitment of MSK1 at inflammatory gene promotors. The curcumin III compositions described herein—and related methods of using such compositions—provide a major step in the transactivation regulation of downstream transcription factors that are key to cell survival and recruitment of inflammatory and immune system events. For example, the ability of the curcumin III compositions described herein to inhibit MSK1 production (or otherwise significantly reduce MSK1 levels) indicates that such compositions may also (indirectly) be used to modulate NFkB (nuclear factor kappa-light-chain-enhancer of activated B cells)—the aberrant expression and transactivation of which has been linked to cancer, inflammation, and autoimmune diseases. The curcumin III compositions (and related methods) of the present invention provide improved efficacy, reliability, and drug target selectivity, relative to natural curcumin extracts.

Still further, the invention provides that certain curcumin- and turmerone-containing compositions of the present invention may be used to modulate BDNF (brain derived neurotrophic factor) expression levels. BDNF plays an important role in neuronal survival and growth (and brain plasticity), which are critical for memory and learning activities. In addition, BDNF is believed to play an important role in certain neurodegenerative diseases. In such embodiments, the composition used to modulate BDNF will preferably comprise at least 30% by weight curcumin II; 30% by weight curcumin III; or 30% by weight curcumin II and curcumin III. In addition, such compositions will preferably include at least 5% by weight of turmerones, such as at least 8%, 10%, 15%, 20%, 30%, or 40% of turmerones.

### Curcumin Compositions

A natural curcumin extract comprises a mixture of curcumin I, desmethoxycurcumin (curcumin II), and bisdemethoxycurcumin (curcumin III). The term curcumin refers to the principal curcuminoid in the Indian spice turmeric plant (a member of Zingiberaceae). The IUPAC name for the curcumin I molecule is (1E,6E)-1,7-Bis(4-hydroxy-3-methoxyphenyl)-1,6-heptadiene-3,5-dione. Although curcumin I may exist in several different tautomeric forms, the enol form is illustrated below.

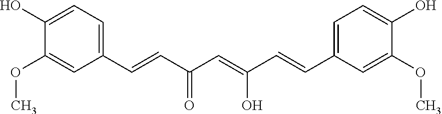 [SMILES: COc1cc(/C=C/C(=O)/C=C(O)/C=C/c2ccc(O)c(OC)c2)ccc1O]

The IUPAC name for the desmethoxycurcumin (curcumin II) molecule is (1E,6E)-1-(4-Hydroxy-3-methoxyphenyl)-7-(4-hydroxyphenyl)hepta-1,6-diene-3,5-dione, and has the chemical structure shown below.

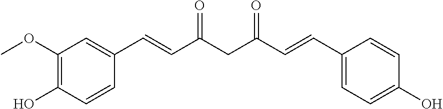 [SMILES: COc1cc(/C=C/C(=O)CC(=O)/C=C/c2ccc(O)cc2)ccc1O]

The IUPAC name for bis-desmethoxycurcumin (curcumin III) that is used in the compositions and methods of the present invention is (1E,6E)-1,7-bis(4-hydroxyphenyl)hepta-1,6-diene-3,5-dione, and has the chemical structure shown below.

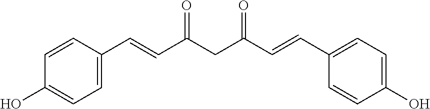 [SMILES: O=C(/C=C/c1ccc(O)cc1)CC(=O)/C=C/c1ccc(O)cc1]

According to certain preferred embodiments, the invention provides that curcumin II and curcumin III may be extracted from turmeric plant rhizome (Curcuma longa) and subsequently concentrated to the desired levels. Alternatively, the invention provides that the curcumin II and curcumin III molecules may be chemically synthesized and used to formulate a therapeutic composition described herein. As explained herein, the desired concentration of curcumin II and/or curcumin III is at least 30%, 50%, or 70% by weight curcumin II; at least 30%, 50%, or 70% by weight curcumin III; or at least 30%, 50%, or 70% by weight curcumin II and curcumin III.

### Turmerone Compositions

The turmerone compositions described herein refer to a group of related compounds of the sesquiterpene class. Such turmerones also exist within the turmeric plant rhizome (Curcuma longa)—and can also be found in related plants, such as Curcuma caesia. The turmerone compositions described herein include alpha-turmerone, beta-turmerone (also known as curlone), ar-turmerone (aromatic turmerone), and a mixture of two or more of such turmerones (and all stereoisomers of the foregoing).

The IUPAC name for alpha-turmerone is 2-methyl-6-(4-methylcyclohexa-2,4-dien-1-yl)hept-2-en-4-one. A chemical structure for alpha-turmerone is reproduced below.

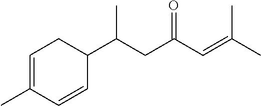 [SMILES: CC(C)=CC(=O)CC(C)C1C=CC(C)=CC1]

The IUPAC name for beta-turmerone (also known as curlone) is 2-methyl-6-(4-methylidenecyclohex-2-en-1-yl)hept-2-en-4-one. A chemical structure for beta-turmerone is reproduced below.

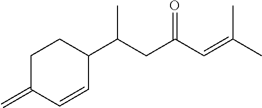 [SMILES: C=C1C=CC(C(C)CC(=O)C=C(C)C)CC1]

The IUPAC name for ar-turmerone is 2-methyl-6-(4-methylphenyl)hept-2-en-4-one. A chemical structure for ar-turmerone is reproduced below.

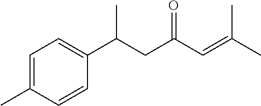 [SMILES: CC(C)=CC(=O)CC(C)c1ccc(C)cc1]

The invention provides that such turmerones may be extracted from turmeric plant rhizome (Curcuma longa) and subsequently concentrated to the desired levels. Alternatively, the invention provides that such turmerone molecules may be chemically synthesized and used to formulate a composition described herein. As explained herein, the desired concentration of such turmerones (one or more of such turmerones) in the compositions of the present invention is at least 5% by weight of turmerones, such as at least 8%, 10%, 15%, 20%, 30%, or 40% by weight of total turmerones.

### Formulations

The invention provides that the compositions described herein may be administered in any desired and effective manner, e.g., as pharmaceutical compositions or nutritional supplements for oral ingestion. More particularly, for example, pharmaceutically acceptable compositions or nutritional supplements of the invention may comprise one or more of the compositions described herein with one or more acceptable carriers. Regardless of the route of administration selected, the compositions may be formulated into acceptable dosage forms by conventional methods known to those of skill in the art. For example, acceptable carriers include, but are not limited to, sugars (e.g., lactose, sucrose, mannitol, and sorbitol), silicon dioxide, starches, cellulose preparations (such as microcrystalline cellulose), calcium phosphates (e.g., dicalcium phosphate, tricalcium phosphate and calcium hydrogen phosphate), sodium citrate, water, aqueous solutions, alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes, paraffins, silicones, talc, silicylate, etc.

Each acceptable carrier used in a pharmaceutical composition or nutritional supplement of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions and nutritional supplements of the invention may, optionally, contain additional ingredients and/or materials commonly used in pharmaceutical compositions and/or nutritional supplements. Such ingredients and materials include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxy methyl cellulose and sodium carbonate; (5) solution retarding agents, such as paraffin; (6) absorption accelerators, such as quaternary ammonium compounds; (7) wetting agents, such as cetyl alcohol and glycerol monosterate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; (28) vitamins and minerals; (29) proteins that carry therapeutic or nutritional benefits, such as whey protein and other milk-derived proteins; and (30) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions and nutritional supplements suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, or a paste. These formulations may be prepared by methods known in the art, e.g., by means of conventional pan-coating, mixing, granulation or lyophilization processes.

Solid dosage forms for oral administration (capsules, tablets, pills, powders, granules and the like) may be prepared by mixing the active ingredient(s) with one or more acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as capsules, pills and granules, may optionally be scored or prepared with coatings and shells, such as enteric coatings and other coatings well known in the art. The tablets, and other solid dosage forms, may also be formulated so as to provide slow or controlled release of the active ingredient therein. They may be sterilized by, for example, filtration through a bacteria-retaining filter. These compositions may also optionally contain opacifying agents that release the active ingredient only, or preferentially, in a certain portion of the gastrointestinal tract, optionally, in a delayed manner. The active ingredient can also be in a microencapsulated form.

Liquid dosage forms for oral administration include acceptable emulsions, microemulsions, solutions, suspensions, syrups, and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

### Methods for Using the Curcumin- and Turmerone-Compositions

According to certain preferred embodiments of the present invention, methods for increasing LDL receptor expression levels (and thereby lowering LDL) in a plurality of cells (and a subject) are provided. In such embodiments, the methods include providing to the cells (or administering to a biological system that comprises a plurality of cells) an effective amount of a curcumin- and turmerone-enriched composition described herein. According to additional preferred embodiments of the present invention, methods for inhibiting MSK1 serine³⁷⁶ phosphorylation in a plurality of cells are provided. Such methods include providing to the cells (or administering to a biological system that comprises a plurality of cells) an effective amount of a curcumin- and turmerone-enriched composition described herein.

The “effective amount” of a LDL receptor-modulating curcumin and turmerone composition will preferably be sufficient to significantly increase LDL receptor expression levels (such as by at least 10% relative to a control cell line or, even more preferably, by at least 20% relative to a control cell line), to thereby reduce the amount of LDL in the target cells (and subject). Similarly, the “effective amount” of a MSK1-modulating curcumin and turmerone composition will preferably be sufficient to significantly reduce the amount of MSK1 protein being expressed in the target cells (such as by at least 10% relative to a control cell line or, even more preferably, by at least 20% relative to a control cell line).

According to certain preferred embodiments of the present invention, methods for preventing and/or ameliorating the effects of certain diseases associated with high cholesterol (LDL) levels are provided. Such methods generally include providing to a subject an effective amount of the curcumin- and turmerone-enriched compositions described herein. Non-limiting examples of such diseases include cardiovascular diseases (including heart disease, stroke, peripheral vascular disease, atherosclerosis, arteriosclerosis, and serum LDL elevation), diabetes, and high blood pressure.

According to yet further preferred embodiments of the present invention, methods for preventing and/or ameliorating the effects of an adverse medical condition in which MSK1 is implicated are provided, including glucocorticoid-resistant inflammatory diseases and chemotherapy-resistant cancers. In such embodiments, the methods include providing to a subject a curcumin- and turmerone-enriched composition described herein. The invention provides that elevated levels of curcumin III will selectively inhibit MSK1 production, which thereby produces desirable anti-inflammatory activity. In addition, the invention provides that the compositions and methods described herein may be used for therapeutic nutrition; anti-inflammatory therapy for autoimmune disease and other chronic and acute inflammatory ailments; treatment of pain, swelling and inflammation; nutritional supplementation; superbug treatments; and antimicrobial, antifungal, antibacterial, and antiviral therapies.

In certain specific embodiments, the compositions and methods described herein may also be used to ameliorate the effects of autoimmune diseases (and other inflammatory conditions), such as rheumatoid arthritis, colitis, non-specific inflammatory bowel diseases, crohn's disease, lupus, multiple sclerosis, psoriasis, type-I diabetes, diabetes, myocarditis, thyroiditis, uveitis, systemic lupus erythromatosis, myasthenia and gravis. Furthermore, the compositions and methods described herein may be used to ameliorate the effects of autoimmune syndromes, such as the sources of immune-mediated inflammation (which can promote chronic inflammation, Alzheimer's, asthma, allergies, obesity, chronic fatigue, fibromyelia, premature aging, and general memory impediments). Still further, the compositions and methods may be used for the purpose of performance enhancement; recovery from physical exercise; and to help neutralize lactic acid, oxidation and associated inflammatory responses to workload to improve recovery rate, anabolism, reduce post-workout soreness and associated fatigue (and allow for repeat workout sessions earlier than could otherwise be executed in typical workout and training cycles).

According to yet further embodiments, the invention provides that certain curcumin- and turmerone-containing compositions of the present invention may be used to modulate BDNF (brain derived neurotrophic factor) expression levels. As mentioned above, BDNF plays an important role in neuronal survival and growth, which are critical for memory and learning activities. In such embodiments, the composition used to modulate BDNF will preferably comprise at least 30% by weight curcumin II; 30% by weight curcumin III; or 30% by weight curcumin II and curcumin III. In addition, such composition will preferably include at least 5% by weight of turmerones, such as at least 8%, 10%, 15%, 20%, 30%, or 40% of turmerones.

### EXAMPLES

Example 1

| Component | Amount |
|---|---|
| Curcumin Powder Extract (Curcuma longa) (Curcumin I, II, | 300 mg |
| and III); Extract is at least 95% Curcumin Extract, with no | |
| less than 30% (w/w) Curcumin III | |
| Turmerone Powder (no less than 11% (w/w) of a mixture of | 50 mg |
| alpha-turmerone, beta-turmerone, and ar-turmerone) | |

Example 2

| Component | Amount |
|---|---|
| Curcumin Powder Extract (Curcuma longa) (Curcumin I, II, | 400 mg |
| and III); Extract is at least 95% Curcumin Extract, with no | |
| less than 30% (w/w) Curcumin III | |
| Turmerone Powder (no less than 11% (w/w) of a mixture of | 100 mg |
| alpha-turmerone, beta-turmerone, and ar-turmerone) | |

Example 3

| Component | Amount |
|---|---|
| Curcumin Powder Extract (Curcuma longa) (Curcumin I, II, | 377 mg |
| and III); Extract is at least 95% Curcumin Extract, with no | |
| less than 30% (w/w) Curcumin III | |
| Turmerone Powder (no less than 11% (w/w) of a mixture of | 100 mg |
| alpha-turmerone, beta-turmerone, and ar-turmerone) | |

Example 4

| Component | Amount |
|---|---|
| Curcumin Powder Extract (Curcuma longa) (Curcumin I, II, | 200 mg |
| and III); Extract is at least 95% Curcumin Extract, with no | |
| less than 30% (w/w) Curcumin III | |
| Turmerone Powder (no less than 11% (w/w) of a mixture of | 100 mg |
| alpha-turmerone, beta-turmerone, and ar-turmerone) | |

Example 5

| Component | Amount |
|---|---|
| Curcumin Powder Extract (Curcuma longa) (Curcumin I, II, | 500 mg |
| and III); Extract is at least 95% Curcumin Extract, with no | |
| less than 30% (w/w) Curcumin III | |
| Turmerone Powder (no less than 11% (w/w) of a mixture of | 100 mg |
| alpha-turmerone, beta-turmerone, and ar-turmerone) | |

Example 6

| Component | Amount |
|---|---|
| Curcumin Powder Extract (Curcuma longa) (Curcumin I, II, | 500 mg |
| and III); Extract is at least 95% Curcumin Extract, with no | |
| less than 45% (w/w) Curcumin III | |
| Turmerone Powder (no less than 11% (w/w) of a mixture of | 100 mg |
| alpha-turmerone, beta-turmerone, and ar-turmerone) | |

Example 7

| Component | Amount |
|---|---|
| Curcumin Powder Extract (Curcuma longa) (Curcumin I, II, | 400 mg |
| and III); Extract is at least 95% Curcumin Extract, with no | |
| less than 45% (w/w) Curcumin III | |
| Turmerone Powder (no less than 11% (w/w) of a mixture of | 50 mg |
| alpha-turmerone, beta-turmerone, and ar-turmerone) | |

Example 8

| Component | Amount |
|---|---|
| Curcumin Powder Extract (Curcuma longa) (Curcumin I, II, | 400 mg |
| and III); Extract is at least 95% Curcumin Extract | |
| Turmerone Powder (no less than 11% (w/w) of a mixture of | 50 mg |
| alpha-turmerone, beta-turmerone, and ar-turmerone) | |

### Example 9

In this Example, the impact of certain curcumin/turmerone combinations on brain derived neurotrophic factor (BDNF) expression levels was examined. T98G cells, a fibroblast-like cell type isolated from human glioblastoma, were treated with test and control compositions for 48 hours, before cell supernatant was harvested and secreted BDNF protein levels were determined by ELISA. The test and control compositions were dissolved in dimethyl sulfoxide (DMSO) solvent.

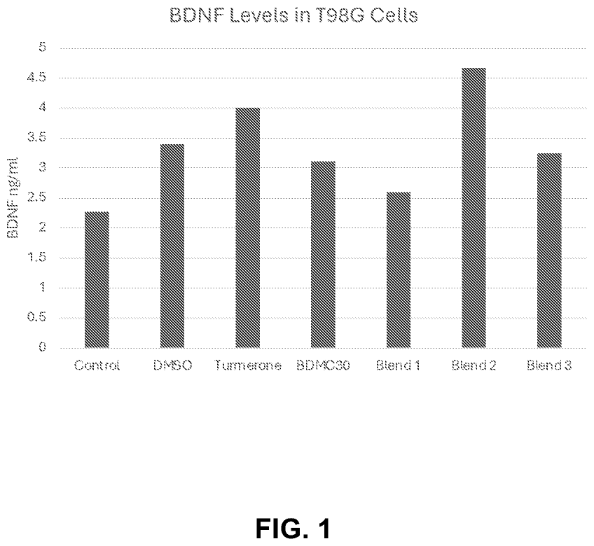

Referring now to FIG. 1, two separate control groups were used, with a first group of cells treated with media alone (“Control”) and a second group of cells treated with media and 0.05% DMSO (“DMSO”). The test groups included cells treated with turmerone alone at a final concentration of 10 μg/ml (“Turmerone”). Another test group was subjected to 10 μg/ml of a curcumin extract known as BDMC30, which contained 30% (w/w) Curcumin III (“BDMC30”). In addition, the test groups included three separate 3:1 curcumin/turmerone blends. Specifically, a first blend combined commercially available curcumin extract and turmerone at a ratio of 3:1, to a final test concentration of 10 μg/ml (“Blend 1”); Blend 2 combined BDMC30 and turmerone at a 3:1 ratio, having a final concentration of 10 μg/ml (“Blend 2”); and a third blend that combined a curcumin extract known as BDMC50 and turmerone at a 3:1 ratio, having a final concentration of 10 μg/ml (“Blend 3”). In Blend 3, BDMC50 contained 50% (w/w) Curcumin III.

All BDNF expression results were normalized to total protein levels for each sample and performed in triplicate. As summarized in FIG. 1, the results demonstrated a significant increase in BDNF expression levels compared to both controls following treatment with Blend 2. Notably, Blend 2 displayed elevated BDNF expression levels compared to turmerone alone and BDMC30 alone, indicating that particular curcumin composition (BDMC30) further increased BDNF expression when combined with turmerone. It was further observed that Blend 3 did not elevate expression as much as Blend 2. It is believed that too much Curcumin II and/or Curcumin III (such as those amounts included in Blend 3 from BDMC50) interfere with normal cell function and the ability to maximize BDNF expression.

The many aspects and benefits of the invention are apparent from the detailed description, and thus, it is intended for the following claims to cover all such aspects and benefits of the invention which fall within the scope and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. A composition that comprises:
(a) a curcumin extract that is supplemented with additional curcumin II, curcumin III, or both curcumin II and curcumin III, such that the composition comprises at least 30% by weight curcumin II, at least 30% by weight curcumin III, or at least 30% by weight of a combination of curcumin II and curcumin III; and
(b) a turmerone composition, such that the composition comprises at least 5% by weight of turmerones.

2. The composition of claim 1, wherein the composition comprises at least 8% by weight of turmerones.

3. The composition of claim 1, wherein the composition comprises at least 10% by weight of turmerones.

4. The composition of claim 1, wherein the composition comprises at least 15% by weight of turmerones.

5. The composition of claim 1, wherein the composition comprises at least 20% by weight of turmerones.

6. The composition of claim 1, wherein the composition comprises at least 30% by weight of turmerones.

7. The composition of claim 1, wherein the composition comprises at least 40% by weight of turmerones.

8. The composition of claim 1, wherein the turmerone composition includes one or more turmerones selected from the group consisting of alpha-turmerone, beta-turmerone, and ar-turmerone.

9. The composition of claim 1, wherein the turmerone composition includes a mixture of alpha-turmerone, beta-turmerone, and ar-turmerone.

10. The composition of claim 1, wherein the composition is formulated as a capsule, pill, tablet, granule, solution or a suspension in an aqueous or non-aqueous liquid, oil-in-water or water-in-oil emulsion, elixir, syrup, paste, or dry powder.

11. The composition of claim 10, wherein the composition is formulated as the capsule, which further comprises hydroxypropyl methylcellulose, water, microcrystalline cellulose, magnesium stearate, and silicon dioxide.

12. A method for increasing expression of brain derived neurotrophic factor (BDNF) in a plurality of cells, which comprises contacting the cells with an effective amount of the composition of claim 1.

13. A method for increasing expression of brain derived neurotrophic factor (BDNF) in a plurality of cells, which comprises contacting the cells with an effective amount of the composition of claim 2.

14. A method for increasing expression of brain derived neurotrophic factor (BDNF) in a plurality of cells, which comprises contacting the cells with an effective amount of the composition of claim 10.

15. A composition that comprises:
(a) a curcumin extract; and
(b) a turmerone composition, such that the composition comprises at least 5% by weight of turmerones.

16. The composition of claim 15, wherein the composition comprises at least 8% by weight of turmerones.

17. The composition of claim 15, wherein the composition comprises at least 10% by weight of turmerones.

18. The composition of claim 15, wherein the composition comprises at least 15% by weight of turmerones.
